(19) United States
(12) Patent Application Publication
(10) Pub. No.: US 2025/0011713 A1
(43) Pub. Date: Jan. 9, 2025

(54) CELL CULTURE DISHES WITH DYNAMICALLY RECONFIGURABLE SURFACE TEXTURES FOR CELL EXPANSION AND HARVESTING

(71) Applicant: University of South Florida, Tampa, FL (US)

(72) Inventors: Nathan GALLANT, Tampa, FL (US); Ryan TOOMEY, Tampa, FL (US); Michael CROSS, Tampa, FL (US)

(21) Appl. No.: 18/763,184

(22) Filed: Jul. 3, 2024

Related U.S. Application Data
(60) Provisional application No. 63/511,743, filed on Jul. 3, 2023.

(51) Int. Cl. C12N 5/00; C12M 1/12

(52) U.S. Cl. CPC C12N 5/0068; C12M 25/04; C12N 2533/30; C12N 2537/10; C12N 2539/10

(57) ABSTRACT

Cell culture substrates that rapidly release cells, including a patterned copolymer including a stimulus-responsive polymer and methods of making and using the same.

### CROSS REFERENCE TO RELATED APPLICATIONS

This application claims the benefit of priority to U.S. Provisional Application No. 63/511,743, filed Jul. 3, 2023, which is incorporated by reference herein in its entirety.

### BACKGROUND

Poly(N-isopropylacrylamide) (PNIPAAm) grafted surfaces have been widely studied for their ability to respond to stimuli present in biological systems leading to the development of new technologies in bioactuation, drug delivery, and wound repair. Limited work has been done with three dimensional PNIPAAm gels.

Unlike grafted PNIPAAm surfaces which undergo 1-dimensional changes, 3D PNIPAAm gel patterns expand both vertically and horizontally. These gels undergo significant changes in volume during the hydrophobic to hydrophilic transition (Akintewe, et al., Shape-Changing Hydrogel Surfaces Trigger Rapid Release of Patterned Tissue Modules. Acta Biomater. 2015, 11, 96-103; Wang, et al., Improving Cracking Resistance of Cement Mortar by Thermo-Sensitive Poly N-Isopropyl Acrylamide (PNIPAM) Gels. J. Clean. Prod. 2018, 176, 1292-1303). One of the major barriers in the wider application of PNIPAAm gels is the inability to control cell adhesion strength to the gels.

Cell adhesion strength can be impacted by many factors including the topography of the surface, surface charge, and the time the cell is adhered to the material (Nguyen, et al., From Nano to Micro: Topographical Scale and Its Impact on Cell Adhesion, Morphology and Contact Guidance. J. Phys. Condens. Matter 2016, 28 (18), 183001; Hallab, et al., Cell Adhesion to Biomaterials: Correlations between Surface Charge, Surface Roughness, Adsorbed Protein, and Cell Morphology. J. Long. Term Eff. Med. Implants 1995, 5 (3), 209-231). Positive surface potential has been shown to increase cell adhesion strength by further spreading the cells allowing more contact area between the cells and the surface (Chang, et al., Effect of Surface Potential on NIH3T3 Cell Adhesion and Proliferation. J. Phys. Chem. C. 2014, 118 (26), 14464-14470).

Several methods have been used to control cell adhesion to PNIPAAm grafted surfaces. Thicker PNIPAAm grafted surfaces have been shown to have decreased cell adhesion due to the expression of vinculin and fibronectin (Lian, et al., Tunable Adhesion of Different Cell Types Modulated by Thermoresponsive Polymer Brush Thickness. Biomacromolecules 2020, 21 (2), 732-742). The introduction of a polyhedral oligomeric silsesquioxane nanoscale inorganic enhanced agent to the NIPAAm matrix has been shown to improve cell adhesion and growth rate on PNIPAAm grafted surfaces (Tong, et al., POSS-Enhanced Thermosensitive Hybrid Hydrogels for Cell Adhesion and Detachment. RSC Adv. 8 (25), 13813-13819). PEGs have been shown to decrease cell adhesion strength. Copolymerization of PNIPAAm with NtBa has been shown to increase cell adhesion by modifying the surface energy (Rochev, et al., Rationalising the Design of Polymeric Thermoresponsive Biomaterials. J. Mater. Sci. Mater. Med. 2004, 15 (4), 513-517). However, controlling cell adhesion has still not been fully realized for responsive cell culture substrates.

Thus, there exists a need for improved cell culture substrates with tunable cell adhesion. This need and others are at least partially satisfied by the present disclosure.

### SUMMARY

The disclosed subject matter is, in one aspect, a cell-culture-supporting surface, such as a cell culture dish, flask, or well plate, with a texture comprised of dynamically reconfigurable features. The surface texture is produced using photolithography methods suitable for large-scale manufacturing. The surface supports growth and maintenance of adherent mammalian cells. Mechanical characteristics of the surface such as size, shape, and arrangement are configurable between states: one that supports adherent cell attachment to one that ejects adhered cells for recovery in a suspension of individual cells for downstream analysis.

In an aspect, provided is a cell culture substrate, including: a support; and a copolymer comprising a stimulus-responsive polymer and an ionic polymer; wherein the copolymer is patterned on the support; and wherein exposure to a stimulus decreases adhesion strength between the cell culture substrate and a plurality of cells seeded on the cell culture substrate, thereby releasing the plurality of cells from the cell culture substrate.

In another aspect, provided is a method of making any of the disclosed cell culture substrates, the method including patterning the copolymer on the support.

In yet another aspect, provided is a method of cell culture, the method including: a) providing any of the disclosed cell culture substrates; b) seeding a plurality of cells on the cell culture substrate; c) exposing the cell culture substrate to a stimulus, thereby releasing the plurality of cells from the cell culture substrate; and d) collecting the plurality of cells.

Other systems, methods, features and/or advantages will be or may become apparent to one with skill in the art upon examination of the following drawings and detailed description. It is intended that all such additional systems, methods, features and/or advantages be included within this description and be protected by the accompanying claims.

### BRIEF DESCRIPTION OF DRAWINGS

FIG. 1 depicts an example of shape-shifting textures for cell ejection.

FIG. 2 depicts an example implementation of reversible adhesion textures.

FIGS. 3A-3B depict PNIPAAm polymer patterns above (FIG. 3A) and below (FIG. 3B) the transition temperature.

FIG. 4 depicts cell adhesion after 24 hours.

FIG. 5 depicts uniform film adhesion strength and patterned film adhesion strength.

FIG. 6 depicts linear regression equations showing tunable adhesion strength.

FIG. 7 depicts the control flask for the detachment experiments.

FIG. 8 depicts cell culture substrates 48 hours after cell seeding.

FIG. 9 depicts the same flasks in FIG. 8 after cell detachment.

FIG. 10 depicts the cells collected from the flasks shown in FIG. 8 24 hours after detachment and reseeding.

### DETAILED DESCRIPTION

It is appreciated that certain features of the disclosure, which are, for clarity, described in the context of separate aspects, can also be provided in combination with a single aspect. Conversely, various features of the disclosure, which are, for brevity, described in the context of a single aspect, can also be provided separately or in any suitable subcombination. Unless defined otherwise, all technical and scientific terms used herein have the same meaning as commonly understood by one of ordinary skill in the art. Methods and materials similar or equivalent to those described herein can be used in the practice or testing of the present disclosure.

### Definitions

In this specification and in the claims that follow, reference will be made to a number of terms, which shall be defined to have the following meanings:

As used herein, “comprising” is to be interpreted as specifying the presence of the stated features, integers, steps, or components as referred to, but does not preclude the presence or addition of one or more features, integers, steps, or components, or groups thereof. Moreover, each of the terms “by”, “comprising,” “comprises”, “comprised of,” “including,” “includes,” “included,” “involving,” “involves,” “involved,” and “such as” are used in their open, non-limiting sense and may be used interchangeably. Further, the term “comprising” is intended to include examples and aspects encompassed by the terms “consisting essentially of” and “consisting of.” Similarly, the term “consisting essentially of” is intended to include examples encompassed by the term “consisting of.

As used in the specification and the appended claims, the singular forms “a,” “an” and “the” include plural referents unless the context clearly dictates otherwise. Thus, for example, reference to “a compound”, “the composition”, or “an agent”, includes, but is not limited to, two or more such compounds, compositions, or agents, and the like.

It should be noted that ratios, concentrations, amounts, and other numerical data can be expressed herein in a range format. It can be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. Ranges can be expressed herein as from “about” one particular value, and/or to “about” another particular value. Similarly, when values are expressed as approximations, by use of the antecedent “about,” it can be understood that the particular value forms a further aspect. For example, if the value “about 10” is disclosed, then “10” is also disclosed.

When a range is expressed, a further aspect includes from the one particular value and/or to the other particular value. For example, where the stated range includes one or both of the limits, ranges excluding either or both of those included limits are also included in the disclosure, e.g. the phrase “x to y” includes the range from ‘x’ to ‘y’ as well as the range greater than ‘x’ and less than ‘y’. The range can also be expressed as an upper limit, e.g. ‘about x, y, z, or less’ and should be interpreted to include the specific ranges of ‘about x’, ‘about y’, and ‘about z’ as well as the ranges of ‘less than x’, less than y′, and ‘less than z’. Likewise, the phrase ‘about x, y, z, or greater’ should be interpreted to include the specific ranges of ‘about x’, ‘about y’, and ‘about z’ as well as the ranges of ‘greater than x’, greater than y′, and ‘greater than z’. In addition, the phrase “about ‘x’ to ‘y’”, where ‘x’ and ‘y’ are numerical values, includes “about ‘x’ to about ‘y’”.

It is to be understood that such a range format is used for convenience and brevity, and thus, should be interpreted in a flexible manner to include not only the numerical values explicitly recited as the limits of the range, but also to include all the individual numerical values or sub-ranges encompassed within that range as if each numerical value and sub-range is explicitly recited. To illustrate, a numerical range of “about 0.1% to 5%” should be interpreted to include not only the explicitly recited values of about 0.1% to about 5%, but also include individual values (e.g., about 1%, about 2%, about 3%, and about 4%) and the sub-ranges (e.g., about 0.5% to about 1.1%; about 5% to about 2.4%; about 0.5% to about 3.2%, and about 0.5% to about 4.4%, and other possible sub-ranges) within the indicated range.

As used herein, the terms “about,” “approximate,” “at or about,” and “substantially” mean that the amount or value in question can be the exact value or a value that provides equivalent results or effects as recited in the claims or taught herein. That is, it is understood that amounts, sizes, formulations, parameters, and other quantities and characteristics are not and need not be exact, but may be approximate and/or larger or smaller, as desired, reflecting tolerances, conversion factors, rounding off, measurement error and the like, and other factors known to those of skill in the art such that equivalent results or effects are obtained. In some circumstances, the value that provides equivalent results or effects cannot be reasonably determined. In such cases, it is generally understood, as used herein, that “about” and “at or about” mean the nominal value indicated ±10% variation unless otherwise indicated or inferred. In general, an amount, size, formulation, parameter or other quantity or characteristic is “about,” “approximate,” or “at or about” whether or not expressly stated to be such. It is understood that where “about,” “approximate,” or “at or about” is used before a quantitative value, the parameter also includes the specific quantitative value itself, unless specifically stated otherwise.

As used herein, the terms “optional” or “optionally” means that the subsequently described event or circumstance can or cannot occur, and that the description includes instances where said event or circumstance occurs and instances where it does not.

### Cell Culture Substrate

In an aspect, provided is a cell culture substrate, including: a support; and a copolymer comprising a stimulus-responsive polymer and an ionic polymer; wherein the copolymer is patterned on the support; and wherein exposure to a stimulus decreases adhesion strength between the cell culture substrate and a plurality of cells seeded on the cell culture substrate, thereby releasing the plurality of cells from the cell culture substrate.

In some aspects, the support can be any cell culture-compatible material which could be identified by one of skill in the art. For example, in some aspects, the support can include glass, metal, quartz, tissue culture plastic, or any combination thereof. In some aspects, the support can be flat or curved.

In some aspects, the stimulus-responsive polymer can include poly(N-isopropylacrylamide) (PNIPAAm), poly(oligo(ethylene glycol) methyl ether methacrylate) (POEGMA), polyethylene oxide (PEO), polyhydroxyethylmethacrylate (PHEMA), poly[2-(dimethylamino)ethyl methacrylate] (PDMAEMA), or any combination thereof.

As used herein, the term “stimulus-responsive polymer” refers to a polymer or copolymer which is chemically or physically modified or altered in response to a stimulus. For example, PNIPAAm is a stimulus-responsive polymer, specifically a thermoresponsive polymer (i.e., chemically or physically modified or altered in response to a change in temperature) that has a Lower Critical Solution Temperature (LCST). This means that the polymer dissolves in solvent (e.g., water), however, when this polymer solution is heated above its LCST, it undergoes a reversible phase transition from a soluble hydrated state to an insoluble dehydrated state. In the case of a hydrogel of PNIPAAm, the gel is collapsed (water expelled) above the LCST and swollen (water absorbed) below it. Other thermoresponsive polymers have an Upper Critical Solution Temperature (UCST), and still others have both an LCST and UCST. Other stimulus-responsive polymers may have other critical points at which they respond to other stimuli (e.g., undergo a reversible phase transition), such as change in pH, an electrical or magnetic field, radiation, ionic concentration, etc. as further discussed below.

In some aspects, the ionic polymer can include a cationic polymer, a zwitterionic polymer, an anionic polymer, or any combination thereof.

In some aspects, the ionic polymer can include n-(3-aminopropyl) methacrylamide hydrochloride (APMA), acrylic acid, [2-(methacryloyloxy)ethyl]dimethyl-(3-sulfopropyl) ammonium hydroxide, or any combination thereof.

In some aspects, the copolymer can include at least about 0.1% (e.g., at least about 0.2%, at least about 0.3%, at least about 0.4%, at least about 0.5%, at least about 1%, at least about 1.5%, at least about 2%, at least about 2.5%, at least about 3%, at least about 3.5%, at least about 4%, at least about 4.5%, at least about 5%, at least about 5.5%, at least about 6%, at least about 6.5%, at least about 7%, at least about 7.5%, at least about 8%, at least about 8.5%, at least about 9%, at least about 9.5%, at least about 10%, at least about 11%, at least about 12%, at least about 13%, at least about 14%, at least about 15%, at least about 16%, at least about 17%, at least about 18%, at least about 19%, at least about 20%) of the ionic polymer. In some aspects, the copolymer can include up to about 20% (e.g., up to about 19%, up to about 18%, up to about 17%, up to about 16%, up to about 15%, up to about 14%, up to about 13%, up to about 12%, up to about 11%, up to about 10%, up to about 9.5%, up to about 9%, up to about 8.5%, up to about 8%, up to about 7.5%, up to about 7%, up to about 6.5%, up to about 6%, up to about 5.5%, up to about 5%, up to about 4.5%, up to about 4%, up to about 3.5%, up to about 3%, up to about 2.5%, up to about 2%, up to about 1.5%, up to about 1%, up to about 0.5%, up to about 0.4%, up to about 0.3%, up to about 0.2%, up to about 0.1%) of the ionic polymer.

It is considered that the copolymer can include an amount of the ionic polymer ranging from any of the minimum values described above to any of the maximum values described above. For example, in some aspects, the copolymer can include from about 0.1% to about 20% (e.g., from about 0.2% to about 19%, from about 0.3% to about 18%, from about 0.4% to about 17%, from about 0.5% to about 16%, from about 1% to about 15%, from about 1.5% to about 14%, from about 2% to about 13%, from about 2.5% to about 12%, from about 3% to about 11%, from about 3.5% to about 10%, from about 4% to about 9.5%, from about 4.5% to about 9%, from about 5% to about 8.5%, from about 5.5% to about 8%, from about 6% to about 7.5%, from about 6.5% to about 7%, from about 0.1% to about 7%, from about 0.2% to about 6.5%, from about 0.3% to about 6%, from about 0.4% to about 5.5%, from about 0.5% to about 5%, from about 1% to about 4.5%, from about 1.5% to about 4%, from about 2% to about 3.5%, from about 2.5% to about 3%, from about 6.5% to about 20%, from about 7% to about 19%, from about 7.5% to about 18%, from about 8% to about 17%, from about 8.5% to about 16%, from about 9% to about 15%, from about 9.5% to about 14%, from about 10% to about 13%, from about 11% to about 12%) of the ionic polymer.

In some aspects, the copolymer can include a photoactivatable crosslinker. In some aspects, the photoactivatable crosslinker can include methacryloyl-4-oxy-benzophenone (MABP), 2,2-dimethoxy-2-phenylacetophenone (DMPA), 2-(4-benzoyl-3-hydroxyphenoxy)ethyl acrylate, or any combination thereof.

In some aspects, the copolymer can include at least about 1% (e.g., at least about 1.5%, at least about 2%, at least about 2.5%, at least about 3%, at least about 3.5%, at least about 4%, at least about 4.5%, at least about 5%, at least about 5.5%, at least about 6%, at least about 6.5%, at least about 7%, at least about 7.5%, at least about 8%, at least about 8.5%, at least about 9%, at least about 9.5%, at least about 10%) of the photoactivatable crosslinker. In some aspects, the copolymer can include up to about 10% (e.g., up to about 9.5%, up to about 9%, up to about 8.5%, up to about 8%, up to about 7.5%, up to about 7%, up to about 6.5%, up to about 6%, up to about 5.5%, up to about 5%, up to about 4.5%, up to about 4%, up to about 3.5%, up to about 3%, up to about 2.5%, up to about 2%, up to about 1.5%, up to about 1%) of the photoactivatable crosslinker

It is considered that the copolymer can include an amount of the photoactivatable crosslinker ranging from any of the minimum values described above to any of the maximum values described above. For example, in some aspects, the copolymer can include from about 1% to about 10% (e.g., from about 1.5% to about 9.5%, from about 2% to about 9%, from about 2.5% to about 8.5%, from about 3% to about 8%, from about 3.5% to about 7.5%, from about 4% to about 7%, from about 4.5% to about 6.5%, from about 5% to about 6%, from about 1% to about 5.5%, from about 1.5% to about 5%, from about 2% to about 4.5%, from about 2.5% to about 4%, from about 3% to about 3.5%, from about 5.5% to about 10%, from about 6% to about 9.5%, from about 6.5% to about 9%, from about 7% to about 8.5%, from about 7.5% to about 8%) of the photoactivatable crosslinker.

In some aspects, the stimulus can be a change in temperature, pH, an electrical or magnetic field, radiation, ionic concentration, or any combination thereof.

In some aspects, the stimulus can be a change in temperature from above a Lower Critical Solution Temperature (LCST) of the copolymer to below said LCST. In other aspects, the stimulus can be a change in temperature of from below an Upper Critical Solution Temperature (UCST) of the copolymer to above said UCST. For example, in some aspects, the stimulus can be a change in temperature of at least about 1° C. (e.g., at least about 2° C., at least about 3° C., at least about 4° C., at least about 5° C., at least about 6° C., at least about 7° C., at least about 8° C., at least about 9° C., at least about 10° C., at least about 11° C., at least about 12° C., at least about 13° C., at least about 14° C., at least about 15° C., at least about 16° C., at least about 17° C., at least about 18° C., at least about 19° C., at least about 20° C.). In some aspects, the stimulus can be a change in temperature of up to about 20° C. (e.g., up to about 19° C., up to about 18° C., up to about 17° C., up to about 16° C., up to about 15° C., up to about 14° C., up to about 13° C., up to about 12° C., up to about 11° C., up to about 10° C., up to about 9° C., up to about 8° C., up to about 7° C., up to about 6° C., up to about 5° C., up to about 4° C., up to about 3° C., up to about 2° C., up to about 1° C.).

It is considered that the stimulus can be a change in temperature ranging from any of the minimum values described above to any of the maximum values described above. For example, in some aspects, the stimulus can be a change in temperature of from about 1° C. to about 20° C. (e.g., from about 2° C. to about 19° C., from about 3° C. to about 18° C., from about 4° C. to about 17° C., from about 5° C. to about 16° C., from about 6° C. to about 15° C., from about 7° C. to about 14° C., from about 8° C. to about 13° C., from about 9° C. to about 12° C., from about 10° C. to about 11° C., from about 1° C. to about 11° C., from about 2° C. to about 10° C., from about 3° C. to about 9° C., from about 4° C. to about 8° C., from about 5° C. to about 7° C., from about 10° C. to about 20° C., from about 11° C. to about 19° C., from about 12° C. to about 18° C., from about 13° C. to about 17° C., from about 14° C. to about 16° C.). It is understood that the change in temperature can refer to a decrease in temperature (e.g., a decrease in temperature of from about 1° C. to about 20° C.) or an increase in temperature (e.g., an increase in temperature of from about 1° C. to about 20° C.).

In some aspects, the stimulus can be a change in pH of at least about 0.1 (e.g., at least about 0.2, at least about 0.3, at least about 0.4, at least about 0.5, at least about 0.6, at least about 0.7, at least about 0.8, at least about 0.9, at least about 1, at least about 1.1, at least about 1.2, at least about 1.3, at least about 1.4, at least about 1.5, at least about 1.6, at least about 1.7, at least about 1.8, at least about 1.9, at least about 2). In some aspects, the stimulus change can be a change in pH of up to about 2 (e.g., up to about 1.9, up to about 1.8, up to about 1.7, up to about 1.6, up to about 1.5, up to about 1.4, up to about 1.3, up to about 1.2, up to about 1.1, up to about 1, up to about 0.9, up to about 0.8, up to about 0.7, up to about 0.6, up to about 0.5, up to about 0.4, up to about 0.3, up to about 0.2, up to about 0.1).

It is considered that the stimulus can be a change in pH ranging from any of the minimum values described above to any of the maximum values described above. For example, in some aspects, the stimulus can be a change in pH of from about 0.1 to about 2 (e.g., from about 0.2 to about 1.9, from about 0.3 to about 1.8, from about 0.4 to about 1.7, from about 0.5 to about 1.6, from about 0.6 to about 1.5, from about 0.7 to about 1.4, from about 0.8 to about 1.3, from about 0.9 to about 1.2, from about 1 to about 1.1, from about 0.1 to about 1, from about 0.2 to about 0.9, from about 0.3 to about 0.8, from about 0.4 to about 0.7, from about 0.5 to about 0.6, from about 1 to about 2, from about 1.1 to about 1.9, from about 1.2 to about 1.8, from about 1.3 to about 1.7, from about 1.4 to about 1.6). It is understood that the change in pH can refer to a decrease in pH (e.g., a decrease in pH of from about 0.1 to about 2) or an increase in pH (e.g., an increase in pH of from about 0.1 to about 2).

In some aspects, surface area and/or volume of the copolymer can increase upon exposure to the stimulus, thereby decreasing adhesion strength between the cell culture substrate and the plurality of cells.

In some aspects, surface area of the copolymer can increase by at least about 0.1% (e.g., at least about 0.2%, at least about 0.3%, at least about 0.4%, at least about 0.5%, at least about 1%, at least about 2%, at least about 3%, at least about 4%, at least about 5%, at least about 10%, at least about 20%, at least about 30%, at least about 40%, at least about 50%, at least about 60%, at least about 70%, at least about 80%, at least about 90%, at least about 100%, at least about 110%, at least about 120%, at least about 130%, at least about 140%, at least about 150%, at least about 160%, at least about 170%, at least about 180%, at least about 190%, at least about 200%) upon exposure to the stimulus. In some aspects, surface area of the copolymer can increase by up to about 200% (e.g., up to about 190%, up to about 180%, up to about 170%, up to about 160%, up to about 150%, up to about 140%, up to about 130%, up to about 120%, up to about 110%, up to about 100%, up to about 90%, up to about 80%, up to about 70%, up to about 60%, up to about 50%, up to about 40%, up to about 30%, up to about 20%, up to about 10%, up to about 5%, up to about 4%, up to about 3%, up to about 2%, up to about 1%, up to about 0.5%, up to about 0.4%, up to about 0.3%, up to about 0.2%, up to about 0.1%) upon exposure to the stimulus.

It is considered that surface area of the copolymer can increase by a percentage ranging from any of the minimum values described above to any of the maximum values described above upon exposure to the stimulus. For example, in some aspects, surface area of the copolymer can increase by from about 0.1% to about 200% (e.g., from about 0.2% to about 190%, from about 0.3% to about 180%, from about 0.4% to about 170%, from about 0.5% to about 160%, from about 1% to about 150%, from about 2% to about 140%, from about 3% to about 130%, from about 4% to about 120%, from about 5% to about 110%, from about 10% to about 100%, from about 20% to about 90%, from about 30% to about 80%, from about 40% to about 70%, from about 50% to about 60%, from about 0.1% to about 60%, from about 0.2% to about 50%, from about 0.3% to about 40%, from about 0.4% to about 30%, from about 0.5% to about 20%, from about 1% to about 10%, from about 2% to about 5%, from about 3% to about 4%, from about 50% to about 200%, from about 60% to about 190%, from about 70% to about 180%, from about 80% to about 170%, from about 90% to about 160%, from about 100% to about 150%, from about 110% to about 140%, from about 120% to about 130%) upon exposure to a stimulus.

In some aspects, volume of the copolymer can increase by at least about 0.1% (e.g., at least about 0.2%, at least about 0.3%, at least about 0.4%, at least about 0.5%, at least about 1%, at least about 2%, at least about 3%, at least about 4%, at least about 5%, at least about 10%, at least about 20%, at least about 30%, at least about 40%, at least about 50%, at least about 60%, at least about 70%, at least about 80%, at least about 90%, at least about 100%, at least about 110%, at least about 120%, at least about 130%, at least about 140%, at least about 150%, at least about 160%, at least about 170%, at least about 180%, at least about 190%, at least about 200%, at least about 210%, at least about 220%, at least about 230%, at least about 240%, at least about 250%, at least about 260%, at least about 270%, at least about 280%, at least about 290%, at least about 300%) upon exposure to the stimulus. In some aspects, volume of the copolymer can increase by up to about 300% (e.g., up to about 290%, up to about 280%, up to about 270%, up to about 260%, up to about 250%, up to about 240%, up to about 230%, up to about 220%, up to about 210%, up to about 200%, up to about 190%, up to about 180%, up to about 170%, up to about 160%, up to about 150%, up to about 140%, up to about 130%, up to about 120%, up to about 110%, up to about 100%, up to about 90%, up to about 80%, up to about 70%, up to about 60%, up to about 50%, up to about 40%, up to about 30%, up to about 20%, up to about 10%, up to about 5%, up to about 4%, up to about 3%, up to about 2%, up to about 1%, up to about 0.5%, up to about 0.4%, up to about 0.3%, up to about 0.2%, up to about 0.1%) upon exposure to the stimulus.

It is considered that the volume of the copolymer can increase by a percentage ranging from any of the minimum values described above to any of the maximum values described above upon exposure to the stimulus. For example, in some aspects, the volume of the copolymer can increase by from about 0.1% to about 300% (e.g., from about 0.2% to about 290%, from about 0.3% to about 280%, from about 0.4% to about 270%, from about 0.5% to about 260%, from about 1% to about 250%, from about 2% to about 240%, from about 3% to about 230%, from about 4% to about 220%, from about 5% to about 210%, from about 10% to about 200%, from about 20% to about 190%, from about 30% to about 180%, from about 40% to about 170%, from about 50% to about 160%, from about 60% to about 150%, from about 70% to about 140%, from about 80% to about 130%, from about 90% to about 120%, from about 100% to about 110%, from about 0.1% to about 100%, from about 0.2% to about 90%, from about 0.3% to about 80%, from about 0.4% to about 70%, from about 0.5% to about 60%, from about 1% to about 50%, from about 2% to about 40%, from about 3% to about 30%, from about 4% to about 20%, from about 5% to about 10%, from about 100% to about 300%, from about 110% to about 290%, from about 120% to about 280%, from about 130% to about 270%, from about 140% to about 260%, from about 150% to about 250%, from about 160% to about 240%, from about 170% to about 230%, from about 180% to about 220%, from about 190% to about 210%) upon exposure to the stimulus.

In some aspects, removing the stimulus can increase adhesion strength between the cell culture substrate and the plurality of cells.

In some aspects, the pattern can include a plurality of elements. In some such aspects, the plurality of elements can include circles, squares, rectangles, hexagons, triangles, or any array or combination thereof. In other such aspects, the plurality of elements can form a grid, stripes, or a concentric pattern.

Each of the plurality of elements can be described by a major lateral dimension. For example, if an element is a circle or square, the major lateral dimension may be the radius, side length, or widest point of the element. As another example, if the pattern includes a grating of parallel stripes or concentric rings, the major lateral dimension may be the width of each stripe or ring. In some aspects, each element can independently have a major lateral dimension of at least about 1 μm (e.g., at least about 2 μm, at least about 3 μm, at least about 4 μm, at least about 5 μm, at least about 10 μm, at least about 15 μm, at least about 20 μm, at least about 25 μm, at least about 30 μm, at least about 35 μm, at least about 40 μm, at least about 45 μm, at least about 50 μm, at least about 55 μm, at least about 60 μm, at least about 65 μm, at least about 70 μm, at least about 75 μm, at least about 80 μm, at least about 85 μm, at least about 90 μm, at least about 95 μm, at least about 100 μm). In some aspects, each element can independently have a major lateral dimension of up to about 100 μm (e.g, up to about 95 μm, up to about 90 μm, up to about 85 μm, up to about 80 μm, up to about 75 μm, up to about 70 μm, up to about 65 μm, up to about 60 μm, up to about 55 μm, up to about 50 μm, up to about 45 μm, up to about 40 μm, up to about 35 μm, up to about 30 μm, up to about 25 μm, up to about 20 μm, up to about 15 μm, up to about 10 μm, up to about 5 μm, up to about 4 μm, up to about 3 μm, up to about 2 μm, up to about 1 μm).

It is considered that each element can independently have a major lateral dimension ranging from any of the minimum values described above to any of the maximum values described above. For example, in some aspects, each element can independently have a major lateral dimension of from about 1 μm to about 100 μm (e.g., from about 2 μm to about 95 μm, from about 3 μm to about 90 μm, from about 4 μm to about 85 μm, from about 5 μm to about 80 μm, from about 10 μm to about 75 μm, from about 15 μm to about 70 μm, from about 20 μm to about 65 μm, from about 25 μm to about 60 μm, from about 30 μm to about 55 μm, from about 35 μm to about 50 μm, from about 40 μm to about 45 μm, from about 1 μm to about 45 μm, from about 2 μm to about 40 μm, from about 3 μm to about 35 μm, from about 4 μm to about 30 μm, from about 5 μm to about 25 μm, from about 10 μm to about 20 μm, from about 40 μm to about 100 μm, from about 45 μm to about 95 μm, from about 50 μm to about 90 μm, from about 55 μm to about 85 μm, from about 60 μm to about 80 μm, from about 65 μm to about 75 μm).

In some aspects, each element can independently have a surface area of at least about 1 μm² (e.g., at least about 2 μm², at least about 3 ρm², at least about 4 μm², at least about 5 μm², at least about 10 μm², at least about 20 μm², at least about 30 μm², at least about 40 μm², at least about 50 μm², at least about 75 μm², at least about 100 μm², at least about 125 μm², at least about 150 μm², at least about 175 μm², at least about 200 μm², at least about 225 μm², at least about 250 μm², at least about 275 μm², at least about 300 μm², at least about 325 μm², at least about 350 μm², at least about 375 μm², at least about 400 μm², at least about 425 μm², at least about 450 μm², at least about 475 μm², at least about 500 μm²). In some aspects, each element can independently have a surface area of up to about 500 μm² (e.g., up to about 475 μm², up to about 450 μm², up to about 425 μm², up to about 400 μm², up to about 375 μm², up to about 350 μm², up to about 325 μm², up to about 300 μm², up to about 275 μm², up to about 250 μm², up to about 225 μm², up to about 200 μm², up to about 175 μm², up to about 150 μm², up to about 125 μm², up to about 100 μm², up to about 75 μm², up to about 50 μm², up to about 40 μm², up to about 30 μm², up to about 20 μm², up to about 10 μm², up to about 5 μm², up to about 4 μm², up to about 3 μm², up to about 2 μm², up to about 1 μm²).

It is considered that each element can independently have a surface area ranging from any of the minimum values described above to any of the maximum values described above. For example, in some aspects, each element can independently have a surface area of from about 1 μm² to about 500 μm² (e.g., from about 2 μm² to about 475 μm², from about 3 μm² to about 450 μm², from about 4 μm² to about 425 μm², from about 5 μm² to about 400 μm², from about 10 μm² to about 375 μm², from about 20 μm² to about 350 μm², from about 30 μm² to about 325 μm², from about 40 μm² to about 300 μm², from about 50 μm² to about 275 μm², from about 75 μm² to about 250 μm², from about 100 μm² to about 225 μm², from about 125 μm² to about 200 μm², from about 150 μm² to about 175 μm², from about 1 μm² to about 175 μm², from about 2 μm² to about 150 μm², from about 3 μm² to about 125 μm², from about 4 μm² to about 100 μm², from about 5 μm² to about 75 μm², from about 10 μm² to about 50 μm², from about 20 μm² to about 40 μm², from about 150 μm² to about 500 μm², from about 175 μm² to about 475 μm², from about 200 μm² to about 450 μm², from about 225 μm² to about 425 μm², from about 250 μm² to about 400 μm², from about 275 μm² to about 375 μm², from about 300 μm² to about 350 μm²).

In some aspects, each element can be spaced apart from adjacent elements by at least about 1 μm (e.g., at least about 2 μm, at least about 3 μm, at least about 4 μm, at least about 5 μm, at least about 10 μm, at least about 15 μm, at least about 20 μm, at least about 25 μm, at least about 30 μm, at least about 35 μm, at least about 40 μm, at least about 45 μm, at least about 50 μm). In some aspects, each element can be spaced apart from adjacent elements by up to about 50 μm (e.g., up to about 45 μm, up to about 40 μm, up to about 35 μm, up to about 30 μm, up to about 25 μm, up to about 20 μm, up to about 15 μm, up to about 10 μm, up to about 5 μm, up to about 4 μm, up to about 3 μm, up to about 2 μm, up to about 1 μm).

It is considered that each element can be spaced apart from adjacent elements by a distance ranging from any of the minimum values described above to any of the maximum values described above. For example, in some aspects, each element can be spaced apart from adjacent element by from about 1 μm to about 50 μm (e.g., from about 2 μm to about 45 μm, from about 3 μm to about 40 μm, from about 4 μm to about 35 μm, from about 5 μm to about 30 μm, from about 10 μm to about 25 μm, from about 15 μm to about 20 μm, from about 1 μm to about 20 μm, from about 2 μm to about 15 μm, from about 3 μm to about 10 μm, from about 4 μm to about 5 μm, from about 15 μm to about 50 μm, from about 20 μm to about 45 μm, from about 25 μm to about 40 μm, from about 30 μm to about 35 μm).

In some aspects, each of the plurality of cells can be in contact with multiple elements.

In some aspects, the pattern can be uniform across the entire cell culture substrate. In other aspects, the pattern can be varied across the cell culture substrate. For example, in some such aspects, the shape, surface area, and/or spacing of the pattern or the elements of the pattern can be varied across the cell culture substrate.

In some aspects, the copolymer can be at least about 0.5 μm thick (e.g., at least about 1 μm thick, at least about 1.5 μm thick, at least about 2 μm thick, at least about 2.5 μm thick, at least about 3 μm thick, at least about 4 μm thick, at least about 5 μm thick, at least about 10 μm, at least about 15 μm thick, at least about 20 μm thick, at least about 25 μm thick, at least about 30 μm thick, at least about 35 μm thick, at least about 40 μm thick, at least about 45 μm thick, at least about 50 μm thick). In some aspects, the copolymer can be up to about 50 μm thick (e.g., up to about 45 μm thick, up to about 40 μm thick, up to about 35 μm thick, up to about 30 μm thick, up to about 25 μm thick, up to about 20 μm thick, up to about 15 μm thick, up to about 10 μm thick, up to about 5 μm thick, up to about 4 μm thick, up to about 3 μm thick, up to about 2.5 μm thick, up to about 2 μm thick, up to about 1.5 μm thick, up to about 1 μm thick, up to about 0.5 μm thick).

It is considered that the copolymer can be a thickness ranging from any of the minimum values described above to any of the maximum values described above. For example, in some aspects, the copolymer can be from about 0.5 μm to about 50 μm thick (e.g., from about 1 μm to about 45 μm thick, from about 1.5 μm to about 40 μm thick, from about 2 μm to about 35 μm thick, from about 2.5 μm to about 30 μm thick, from about 3 μm to about 25 μm thick, from about 4 μm to about 20 μm thick, from about 5 μm to about 15 μm thick, from about 0.5 μm to about 10 μm thick, from about 1 μm to about 5 μm thick, from about 1.5 μm to about 4 μm thick, from about 2 μm to about 3 μm thick, from about 10 μm to about 50 μm thick, from about 15 μm to about 45 μm thick, from about 20 μm to about 40 μm thick, from about 25 μm to about 35 μm thick).

In some aspects, the pattern can include regions with no copolymer, and the plurality of cells can be in contact with both the support and the copolymer.

In some aspects, the copolymer can cover at least about 1% (e.g., at least about 2%, at least about 3%, at least about 4%, at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, about 100%) of the support. In some aspects, the copolymer can cover up to about 100% (e.g., up to about 95%, up to about 90%, up to about 85%, up to about 80%, up to about 75%, up to about 70%, up to about 65%, up to about 60%, up to about 55%, up to about 50%, up to about 45%, up to about 40%, up to about 35%, up to about 30%, up to about 25%, up to about 20%, up to about 15%, up to about 10%, up to about 5%, up to about 4%, up to about 3%, up to about 2%, up to about 1%) of the support.

It is considered that the copolymer can cover a percentage of the support ranging from any of the minimum values described above to any of the maximum values described above. For example, in some aspects, the copolymer can cover from about 1% to about 100% (e.g., from about 2% to about 95%, from about 3% to about 90%, from about 4% to about 85%, from about 5% to about 80%, from about 10% to about 75%, from about 15% to about 70%, from about 20% to about 65%, from about 25% to about 60%, from about 30% to about 55%, from about 35% to about 50%, from about 40% to about 45%, from about 1% to about 45%, from about 2% to about 40%, from about 3% to about 35%, from about 4% to about 30%, from about 5% to about 25%, from about 10% to about 20%, from about 40% to about 100%, from about 45% to about 95%, from about 50% to about 90%, from about 55% to about 85%, from about 60% to about 80%, from about 65% to about 75%) of the support.

In some aspects, the cell culture substrate can further include a uniform layer of the copolymer disposed on the support, and the copolymer can be patterned on the uniform layer.

In some aspects, the uniform layer can be at least about 0.5 μm thick (e.g., at least about 1 μm thick, at least about 1.5 μm thick, at least about 2 μm thick, at least about 2.5 μm thick, at least about 3 μm thick, at least about 4 μm thick, at least about 5 μm thick, at least about 10 μm, at least about 15 μm thick, at least about 20 μm thick, at least about 25 μm thick, at least about 30 μm thick, at least about 35 μm thick, at least about 40 μm thick, at least about 45 μm thick, at least about 50 μm thick). In some aspects, the uniform layer can be up to about 50 μm thick (e.g., up to about 45 μm thick, up to about 40 μm thick, up to about 35 μm thick, up to about 30 μm thick, up to about 25 μm thick, up to about 20 μm thick, up to about 15 μm thick, up to about 10 μm thick, up to about 5 μm thick, up to about 4 μm thick, up to about 3 μm thick, up to about 2.5 μm thick, up to about 2 μm thick, up to about 1.5 μm thick, up to about 1 μm thick, up to about 0.5 μm thick).

It is considered that the uniform layer can be a thickness ranging from any of the minimum values described above to any of the maximum values described above. For example, in some aspects, the uniform layer can be from about 0.5 μm to about 50 μm thick (e.g., from about 1 μm to about 45 μm thick, from about 1.5 μm to about 40 μm thick, from about 2 μm to about 35 μm thick, from about 2.5 μm to about 30 μm thick, from about 3 μm to about 25 μm thick, from about 4 μm to about 20 μm thick, from about 5 μm to about 15 μm thick, from about 0.5 μm to about 10 μm thick, from about 1 μm to about 5 μm thick, from about 1.5 μm to about 4 μm thick, from about 2 μm to about 3 μm thick, from about 10 μm to about 50 μm thick, from about 15 μm to about 45 μm thick, from about 20 μm to about 40 μm thick, from about 25 μm to about 35 μm thick).

In some aspects, the plurality of cells can be mammalian. In some such aspects, the plurality of cells can be human.

It is understood that, unless otherwise noted, any dimensions or physical descriptors of the cell culture substrate (e.g., percent coverage of the copolymer, element size, etc.) described herein refer to said dimensions or physical descriptors before exposure to the stimulus.

### Methods

In an aspect, provided is a method of making any of the disclosed cell culture substrates, the method including patterning the copolymer on the support.

In some aspects, when the copolymer includes a photoactivatable crosslinker, the method can include: a) depositing the copolymer on the support in a uniform layer; b) disposing a patterning mask over the copolymer; c) activating the photoactivatable crosslinker with a light source, thereby crosslinking the copolymer only in regions not covered by the patterning mask; and d) removing any uncrosslinked polymer.

In some aspects, the copolymer can be spin coated on the support in step a).

In some aspects, the light source can be a UV light.

In some aspects, the method can include depositing the copolymer on the support via additive manufacturing (e.g., 3D printing).

In another aspect, provided is a method of cell culture, the method including: a) providing any of the disclosed cell culture substrates; b) seeding a plurality of cells on the cell culture substrate; c) exposing the cell culture substrate to a stimulus, thereby releasing the plurality of cells from the cell culture substrate; and d) collecting the plurality of cells.

In some aspects, the stimulus can be a change in temperature, pH, an electrical or magnetic field, radiation, ionic concentration, or any combination thereof.

In some aspects, the stimulus can be a change in temperature from above a Lower Critical Solution Temperature (LCST) of the copolymer to below said LCST. In other aspects, the stimulus can be a change in temperature of from below an Upper Critical Solution Temperature (UCST) of the copolymer to above said UCST. For example, in some aspects, the stimulus can be a change in temperature of at least about 1° C. (e.g., at least about 2° C., at least about 3° C., at least about 4° C., at least about 5° C., at least about 6° C., at least about 7° C., at least about 8° C., at least about 9° C., at least about 10° C., at least about 11° C., at least about 12° C., at least about 13° C., at least about 14° C., at least about 15° C., at least about 16° C., at least about 17° C., at least about 18° C., at least about 19° C., at least about 20° C.). In some aspects, the stimulus can be a change in temperature of up to about 20° C. (e.g., up to about 19° C., up to about 18° C., up to about 17° C., up to about 16° C., up to about 15° C., up to about 14° C., up to about 13° C., up to about 12° C., up to about 11° C., up to about 10° C., up to about 9° C., up to about 8° C., up to about 7° C., up to about 6° C., up to about 5° C., up to about 4° C., up to about 3° C., up to about 2° C., up to about 1° C.).

It is considered that the stimulus can be a change in temperature ranging from any of the minimum values described above to any of the maximum values described above. For example, in some aspects, the stimulus can be a change in temperature of from about 1° C. to about 20° C. (e.g., from about 2° C. to about 19° C., from about 3° C. to about 18° C., from about 4° C. to about 17° C., from about 5° C. to about 16° C., from about 6° C. to about 15° C., from about 7° C. to about 14° C., from about 8° C. to about 13° C., from about 9° C. to about 12° C., from about 10° C. to about 11° C., from about 1° C. to about 11° C., from about 2° C. to about 10° C., from about 3° C. to about 9° C., from about 4° C. to about 8° C., from about 5° C. to about 7° C., from about 10° C. to about 20° C., from about 11° C. to about 19° C., from about 12° C. to about 18° C., from about 13° C. to about 17° C., from about 14° C. to about 16° C.). It is understood that the change in temperature can refer to a decrease in temperature (e.g., a decrease in temperature of from about 1° C. to about 20° C.) or an increase in temperature (e.g., an increase in temperature of from about 1° C. to about 20° C.).

In some aspects, the stimulus can be a change in pH of at least about 0.1 (e.g., at least about 0.2, at least about 0.3, at least about 0.4, at least about 0.5, at least about 0.6, at least about 0.7, at least about 0.8, at least about 0.9, at least about 1, at least about 1.1, at least about 1.2, at least about 1.3, at least about 1.4, at least about 1.5, at least about 1.6, at least about 1.7, at least about 1.8, at least about 1.9, at least about 2). In some aspects, the stimulus change can be a change in pH of up to about 2 (e.g., up to about 1.9, up to about 1.8, up to about 1.7, up to about 1.6, up to about 1.5, up to about 1.4, up to about 1.3, up to about 1.2, up to about 1.1, up to about 1, up to about 0.9, up to about 0.8, up to about 0.7, up to about 0.6, up to about 0.5, up to about 0.4, up to about 0.3, up to about 0.2, up to about 0.1).

It is considered that the stimulus can be a change in pH ranging from any of the minimum values described above to any of the maximum values described above. For example, in some aspects, the stimulus can be a change in pH of from about 0.1 to about 2 (e.g., from about 0.2 to about 1.9, from about 0.3 to about 1.8, from about 0.4 to about 1.7, from about 0.5 to about 1.6, from about 0.6 to about 1.5, from about 0.7 to about 1.4, from about 0.8 to about 1.3, from about 0.9 to about 1.2, from about 1 to about 1.1, from about 0.1 to about 1, from about 0.2 to about 0.9, from about 0.3 to about 0.8, from about 0.4 to about 0.7, from about 0.5 to about 0.6, from about 1 to about 2, from about 1.1 to about 1.9, from about 1.2 to about 1.8, from about 1.3 to about 1.7, from about 1.4 to about 1.6). It is understood that the change in pH can refer to a decrease in pH (e.g., a decrease in pH of from about 0.1 to about 2) or an increase in pH (e.g., an increase in pH of from about 0.1 to about 2).

In some aspects, at least about 50% (e.g., at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, at least about 99%, about 100%) of the plurality of cells can be released. In some aspects, up to about 100% (e.g., up to about 99%, up to about 98%, up to about 97%, up to about 96%, up to about 95%, up to about 94%, up to about 93%, up to about 92%, up to about 91%, up to about 90%, up to about 85%, up to about 80%, up to about 75%, up to about 70%, up to about 65%, up to about 60%, up to about 55%, up to about 50%) of the plurality of cells can be released.

It is considered that a percentage of the plurality of cells ranging from any of the minimum values described above to any of the maximum values described above can be released. For example, in some aspects, from about 50% to about 100% (e.g., from about 55% to about 99%, from about 60% to about 98%, from about 65% to about 97%, from about 70% to about 96%, from about 75% to about 95%, from about 80% to about 94%, from about 85% to about 93%, from about 90% to about 92%, from about 50% to about 90%, from about 55% to about 85%, from about 60% to about 80%, from about 65% to about 75%, from about 90% to about 100%, from about 91% to about 99%, from about 92% to about 98%, from about 93% to about 97%, from about 94% to about 96%) of the plurality of cells can be released.

In some aspects, the plurality of cells can be released in at least about 1 second (e.g., at least about 2 seconds, at least about 3 seconds, at least about 4 seconds, at least about 5 seconds, at least about 10 seconds, at least about 20 seconds, at least about 30 seconds, at least about 40 seconds, at least about 50 seconds, at least about 60 seconds, at least about 70 seconds, at least about 80 seconds, at least about 90 seconds, at least about 100 seconds, at least about 110 seconds, at least about 120 seconds, at least about 130 seconds, at least about 140 seconds, at least about 150 seconds, at least about 160 seconds, at least about 170 seconds, at least about 180 seconds, at least about 190 seconds, at least about 200 seconds, at least about 210 seconds, at least about 220 seconds, at least about 230 seconds, at least about 240 seconds, at least about 250 seconds, at least about 260 seconds, at least about 270 seconds, at least about 280 seconds, at least about 290 seconds, at least about 300 seconds). In some aspects, the plurality of cells can be released in up to about 300 seconds (e.g., up to about 290 seconds, up to about 280 seconds, up to about 270 seconds, up to about 260 seconds, up to about 250 seconds, up to about 240 seconds, up to about 230 seconds, up to about 220 seconds, up to about 210 seconds, up to about 200 seconds, up to about 190 seconds, up to about 180 seconds, up to about 170 seconds, up to about 160 seconds, up to about 150 seconds, up to about 140 seconds, up to about 130 seconds, up to about 120 seconds, up to about 110 seconds, up to about 100 seconds, up to about 90 seconds, up to about 80 seconds, up to about 70 seconds, up to about 60 seconds, up to about 50 seconds, up to about 40 seconds, up to about 30 seconds, up to about 20 seconds, up to about 10 seconds, up to about 5 seconds, up to about 4 seconds, up to about 3 seconds, up to about 2 seconds, up to about 1 second).

It is considered that the plurality of cells can be released in a duration ranging from any of the minimum values described above to any of the maximum values described above. For example, in some aspects, the plurality of cells can be released in from about 1 second to about 300 seconds (e.g., from about 2 seconds to about 290 seconds, from about 3 seconds to about 280 seconds, from about 4 seconds to about 270 seconds, from about 5 seconds to about 260 seconds, from about 10 seconds to about 250 seconds, from about 20 seconds to about 240 seconds, from about 30 seconds to about 230 seconds, from about 40 seconds to about 220 seconds, from about 50 seconds to about 210 seconds, from about 60 seconds to about 200 seconds, from about 70 seconds to about 190 seconds, from about 80 seconds to about 180 seconds, from about 90 seconds to about 170 seconds, from about 100 seconds to about 160 seconds, from about 110 seconds to about 150 seconds, from about 120 seconds to about 140 seconds, from about 1 second to about 130 seconds, from about 2 seconds to about 120 seconds, from about 3 seconds to about 110 seconds, from about 4 seconds to about 100 seconds, from about 5 seconds to about 90 seconds, from about 10 seconds to about 80 seconds, from about 20 seconds to about 70 seconds, from about 30 seconds to about 60 seconds, from about 40 seconds to about 50 seconds, from about 130 seconds to about 300 seconds, from about 140 seconds to about 290 seconds, from about 150 seconds to about 280 seconds, from about 160 seconds to about 270 seconds, from about 170 seconds to about 260 seconds, from about 180 seconds to about 250 seconds, from about 190 seconds to about 240 seconds, from about 200 seconds to about 230 seconds, from about 210 seconds to about 220 seconds).

In some aspects, from about 50% to about 100% of the plurality of cells (e.g., as described above) can be released in from about 1 second to about 300 seconds (e.g., as described above).

In some aspects, the method can further include: e) reseeding the plurality of cells on a different substrate.

### EXAMPLES

### Example 1: Cell Tissue Culture Support with Dynamically Reconfigurable Surface Textures that Expel Individual and Sparse Cell Populations

Disclosed herein is a cell culture supporting surface (e.g., dish, flask) that has a texture that detaches attached cells of any density (sparse to confluent) upon stimulation of a reconfiguration of the texture.

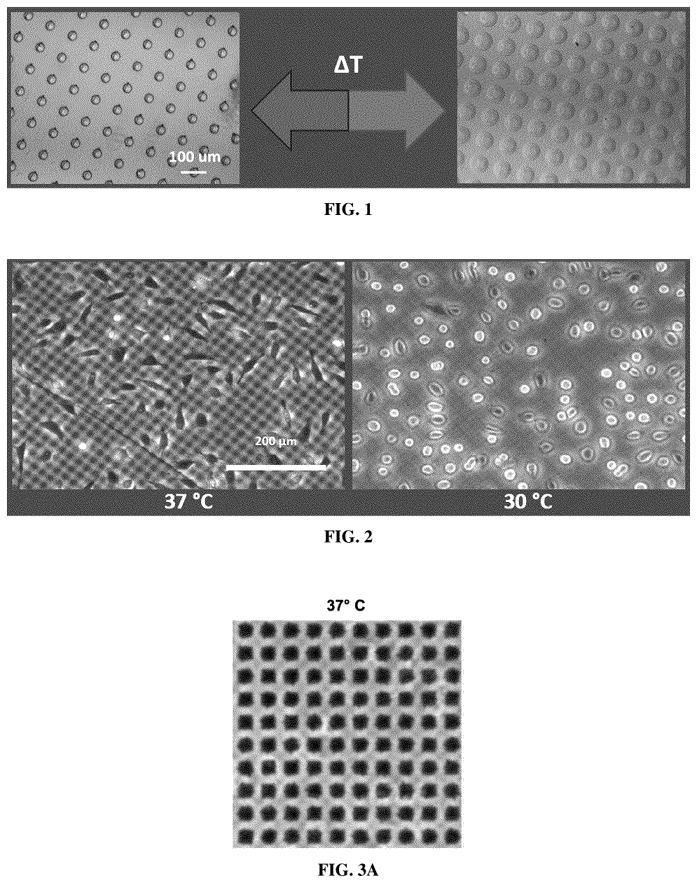

The physical topography of the patterned photoresist (size, shape, and arrangement) can transform between two or more configurations, thereby switching from one that supports stable adhesion to one that ejects adhered cells to recover a suspension of individual cells for downstream analysis. This is shown, for example, in FIG. 1. The non-enzymatic rapid release of cell cultures is non-destructive to cell surface proteins and can release sub-confluent cell monolayers.

Unlike existing technologies that release cell sheets (i.e., large populations of connected cells and matrix detach as monoliths), individual or sparse cell populations are rapidly expelled from this reconfiguring texture.

Ejection of adhered cells is achieved rapidly during texture reconfiguration, which breaks specific non-covalent bonds between cell surface receptors to the patterned photoresist.

The texture includes an array of protruding features which may be of various shapes (circle, square, hexagon, etc.) but critically the dimensions of the features and spaces between features are much smaller than the dimensions of an adhered cell. Thus, a cell may be adhered over many features.

The lateral and spacing dimensions of the texture pattern could be nano- to millimeter scale but generally would be micrometers which is approximately 10-100 times smaller than the length of spread cells. The height of the features is generally equal to or less than the lateral dimensions, but generally of the same order of magnitude. These critical dimensions ensure two essential qualities that trigger rapid detachment: the lateral length to height ratio (along with swelling ratio and shape) modulates the deformation, and the small size (relative to a spreading cell) ensures that multiple features are disrupting the adhesive interface beneath each cell.

The texture is made of a patterned photoresist. The growth surface can be produced with existing photolithography methods for large scale manufacturing. The patterned photoresist supports cell adhesion, growth, and maintenance.

The photoresist is a photocrosslinking polymer that forms an insoluble network when exposed to light. A developer is used to remove unexposed regions to reveal the texture. The photoresist is composed of a stimuli-responsive polymer or co-polymer system such as PNIPAAm, OEGMA, etc., copolymerized with a benzophenone derivative monomer. Excitation of benzophenone at moderate wavelengths produces stable covalent cross-links within the photoresist and between the photoresist and to the supporting culture dish

On textured surfaces with this combination of properties, cells are harvested with greater viable yield from the surface without the use of destructive methods such as enzymes or scraping which can lyse cell surface proteins and break cell membranes. Moreover, the dispersed nature of the harvested suspension of individual cells results in populations of cells ready for subcultivation or assay, in contrast to cell sheets.

The texture is patterned on a culture surface (e.g., polystyrene or glass) by various methods including photolithography or printing.

This reconfigurable surface could be embodied as any culture vessel including a flask, dish, well plate, or planar slide format.

The stimuli-responsive texture could be applied to a flat or curved surface.

The external stimulus could be a change in temperature or pH, an applied electrical or magnetic field, or radiation such as visible or ultraviolet light.

The stimulus does not dissolve the pattern because of physical or chemical crosslinks.

The pattern can be uniform across the culture surface or regional, and the pattern can be varied to organize the cells by contact guidance or encourage random distribution.

The resist may include chemical groups to regulate adsorption of proteins or biological molecules to enhance cell adhesion prior to release. For example, copolymerization with a cation (e.g., 3-aminopropylmethacrylate) enhances increases adsorption of serum proteins including fibronectin and enhances adhesion strength on the texture.

As an example, a grid of 5 micrometer squares that are 2 micrometers tall is patterned onto a polystyrene dish by spin coating a NIPAAm-benzophenone copolymer photoresist and then exposing the film to UV light through a lithographic mask. After removing uncrosslinked resist regions with the developing solvent, the pattern will transform by swelling or collapsing around the transition temperature in aqueous medium. At mammalian cell culture temperature (37° C.), the pattern is in the collapsed (as made) configuration. Upon lowering the temperature to ˜32° C., the patterned squares will expand and deform anisotropically as they swell because they remain linked to the underlying surface at their base. Cells seeded onto this dish will adhere and spread in a random distribution dispersed over the surface. Placing the dish on ice for approximately one minute triggers the volume phase change in the texture. The strain of the swelling feature surfaces breaks the adhesive bonds between the cell and the surface, and the cells round up and detach from the surface. FIG. 2 shows a similar substrate with 10 μm squares spaces 10 μm apart. In the collapsed state, the substrate can support cell attachment. Placing the substrate on ice for 1 minute (e.g., cooling from 37° C. to 30° C.) expands the texture features by about 20% laterally. The rounded cell morphology indicates cell detachment.

The cells grown on this texture at any density are harvested as a dispersed suspension with greater viable yield from the surface without the use of destructive methods such as enzymes or scraping which can lyse cell surface proteins and break cell membranes. Thus, the cells are recovered as a suspension of individual cells for downstream analysis in an undamaged state. Stimuli-responsive surfaces have not been used to harvest individual cells, and patterned surfaces that release cell sheets have dimensions much greater than single cell size.

### Example 2: Copolymerization with Cationic Monomers Enhances Cell Adhesion on Photopatterned PNIPAAm Hydrogels

Prior research on PNIPAAm gels showed that the adhesion strength of cells to PNIPAAm could be increased from 5% to 60% through the incorporation of non-adhesive electrospun fibers (Muñiz Maisonet, et al., Combining Nonadhesive Materials into Microstructured Composite Surfaces Induces Cell Adhesion and Spreading. ACS Biomater. Sci. Eng. 2015, 1 (11), 1163-1173). It had also been observed that coating the surfaces of PNIPAAm gels with lysine increased cell adhesion due to increasing the surface charge of the PNIPAAm.

Micro-scale beams and patterns of PNIPAAm gels are being used for modular tissue engineering. These beams and patterns undergo significant structural changes when swelling. This property is being used to develop technology to release tissue modules and individual cells. The impact of these patterns on cell adhesion strength and growth have not been widely studied.

Based on the observation that cell adhesion strength to PNIPAAm gels was increased by coating the surface with lysine and the prior research showing copolymerization of PNIPAAm with NtBa increases cell adhesion, a study was conducted which hypothesized that the incorporation of cationic monomers into the NIPAAm network would increase cell adhesion to the PNIPAAm surface. The study also tested the impact of patterning the PNIPAAm surfaces on cell adhesion strength. The spinning disk method was used to quantitatively measure the adhesion based on the quantity of cationic monomers in the NIPAAm network.

### Materials and Methods

PNIPAAm Synthesis and Characterization: UV-crosslinkable PNIPAAm samples copolymerized with 0%, 1%, and 5% n-(3-aminopropyl) methacrylamide hydrochloride (APMA) were synthesized. N-Isopropylacrylamide (NIPAM) was combined with 3% Methacryloyl-4-oxy-benzophenone (MABP), 1% Azobisisobutyronitrile (AIBN), and 0%, 1%, and 5% percent APMA and dissolved in 10-20 ml of dimethylformamide (DMF) in a Schlenk tube.

The solution was immersed in liquid nitrogen until frozen then placed under a vacuum for 30 seconds. The sample was then allowed to melt. This freeze, vacuum, thaw cycle was completed until no more gasses could be seen bubbling up from the solution. The cycle was then completed one additional time to ensure as much oxygen was removed as possible. The Schlenk tube was then filled with nitrogen. The top of the Schlenk tube was wrapped with paraffin paper and placed in a 70° C. water bath overnight to activate the AIBN.

A 5-inch piece of dialysis tubing was immersed in distilled water for one minute to soften. The tubing was then clamped on one end with a dialysis clamp. The sample was poured from the Schlenk tube into the dialysis tubing and the other end of the tube was clamped. The sample was then submerged in distilled water. The water was changed every 3 hours for a period of 9 hours then left in fresh distilled water overnight.

Lyophilization was used to remove the water from the sample. The polymer solution was placed into two 100 ml tubes and frozen. The sample was then lyophilized for a period of 1 to 3 days until all water was removed.

APMA slows the reaction resulting in a lower polymer yield. 0% APMA PNIPAAm had a 94% yield, 1% APMA had a 64% yield, and 5% APMA had a 55% yield.

GPC and NMR spectroscopy (Avance NEO 400 MHZ) were used to characterize the polymers. GPC results showed an increased dispersity for polymers with higher APMA concentrations which is consistent with the slowed reaction.

Thin Film Fabrication and Analysis: Circular glass coverslips were immersed in ethanol and placed in a sonication bath for 20 minutes. The cover slips were removed from the bath and dried with pressurized air then placed in the plasma cleaner for one 5 minute cycle.

The cover slips were placed face up in a 1% solution of (3-Aminopropyl)triethoxysilane (APTES) in acetone for two minutes. The cover slips were then rinsed in acetone and blown dry with pressurized nitrogen. Cover slips were placed in the oven at 120 C for 15 minutes.

Synthesized PNIPAAm polymers of varying percent APMA were dissolved in cyclohexanone to make a 25.3% solution.

Cover slips were blown with pressurized nitrogen to remove any dust and placed on a spin coating machine. The polymer solution was pipetted onto the cover slip. The spin coating machine was spun at 2,000 rpm for 75 seconds to create ˜ 1,000 nm thin films. The polymer films were then baked in the oven at 120° C. for 30 minutes to fully dry.

Uniform polymer layers were created by placing the spin coated films 2 cm below a 365 nm UV light. Patterned thin films were created using photolithography. A square patterned mask with 100 μm² features was placed face down between the UV light and the polymer film. The UV light was placed 2 cm above the spin coated films. The films were then rinsed in cyclohexanone to remove non crosslinked polymers.

Cell Culture: The polymer thin films were immersed in 70% ethanol and cooled to below the transition temperature. The thin films were then warmed to above the transition temperature and the ethanol was aspirated. This process was repeated one time. The polymer thin films were then submerged in PBS, cooled to below the transition temperature, then warmed to above the transition temperature. This process was repeated once to ensure all ethanol had been removed. The thin films were placed in a solution of DMEM, 10% penicillin, and 10% newborn calf serum and left in the incubator overnight.

NIH3T3 cells were cultured on polystyrene dishes in the DMEM, streptomycin, and NCS solution. The cells were removed from the tissue culture plates using Trypsin/EDTA. Cells were seeded on the cleaned and warmed polymer thin films and placed in the incubator to adhere for 24 hours.

Cell Adhesion Strength Assay: A spinning disk machine was used to apply a well-defined range of hydrodynamic forces to the cells [18], [19]. The sample was mounted on the spinning disk machine and submerged in a solution of 2 mM of dextrose in DPBS. The spinning disk machine had a programmable heating element that was used to control the temperature during the experiment.

The linear variability of the applied shear stress τ=force/area is shown in the formula below. r is the radial position along the substrate, ρ is the solution density, μ is the solution viscosity, and ω is the angular velocity.

τ=0.8r(ρμω³)^{0.5}

The samples were each spun for a period of 5 minutes. The samples were then fixed to the surface using a 3.7% solution of formaldehyde in PBS, permeabilized using a 0.1% solution of Triton X-100 in pbs, and stained with fluorescent dye.

Automated microscopy was used to count remaining cells adhered at 61 positions on each sample. The counts were used to develop a detachment profile (f vs. τ) which was fit to a sigmoid curve. The curve was used to determine the strength τ₅₀ (shear stress required for 50% cell detachment).

$$f = \frac{1}{1 + {e}^{b ( τ - {τ}_{50} )}}$$

### Results and Discussion

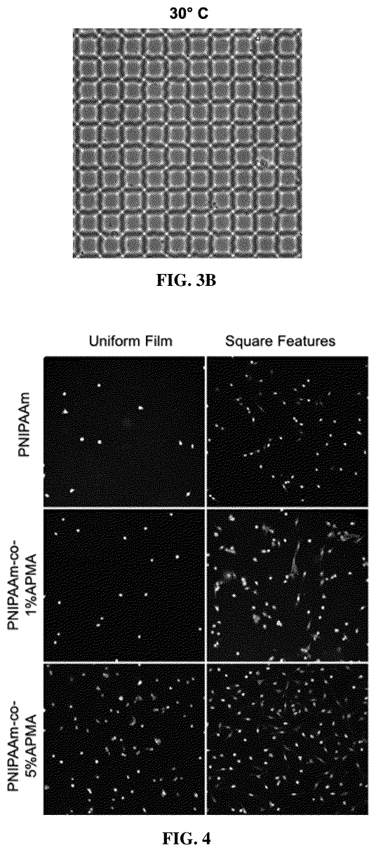

Cell Seeding and Attachment to Uniform and Patterned Surfaces: Using a thermoresponsive polymer, we produced patterns that display a significant change in volume from collapsed (FIG. 3A) to swollen (FIG. 3B) states. Cell adhesion to uniform PNIPAAm films is poor, however, the study showed that enhance adhesion strength can be enhanced by including APMA and square features. This enhancement in adhesion strength is shown by the further spreading of cells in FIG. 4.

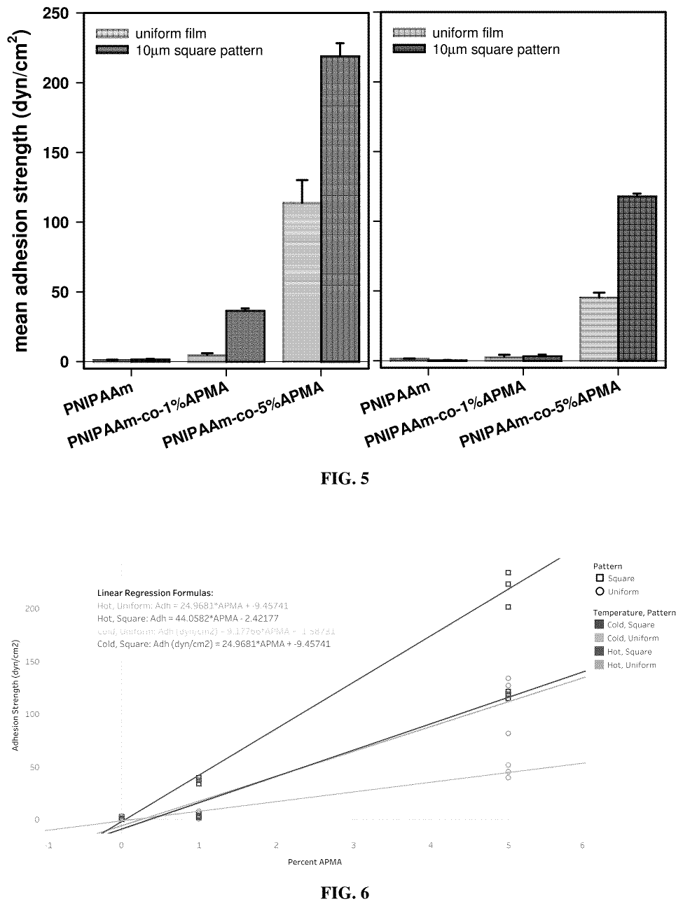

APMA Impact on PNIPAAm Film Adhesion Strength: A significant increase in cell adhesion strength was found due to the incorporation of APMA. At 37° C., uniform PNIPAAm (0% 3-APMA) films showed an average adhesion strength of 1.3 dyn/cm², while adding 3-APMA increased the adhesion strength 3.6-fold and 88-fold for 1% and 5%, respectively. Adhesion strength was further enhanced on textured surfaces compared to uniform films with and without the addition of the cationic monomer. Adhesion strength was nearly 200-fold greater on the patterned 5% 3-APMA relative to the uniform PNIPAAm surface. This is shown in FIG. 5.

Below the transition temperature (25° C.), similar trends were observed (increases with cation and texture); however, the magnitudes of the adhesion strength enhancements were consistently lower compared to 37° C. Importantly, the difference in cell adhesion strength between conditions above and below the transition temperature also increased as the amount of 3-APMA increased as the adhesion strength increased.

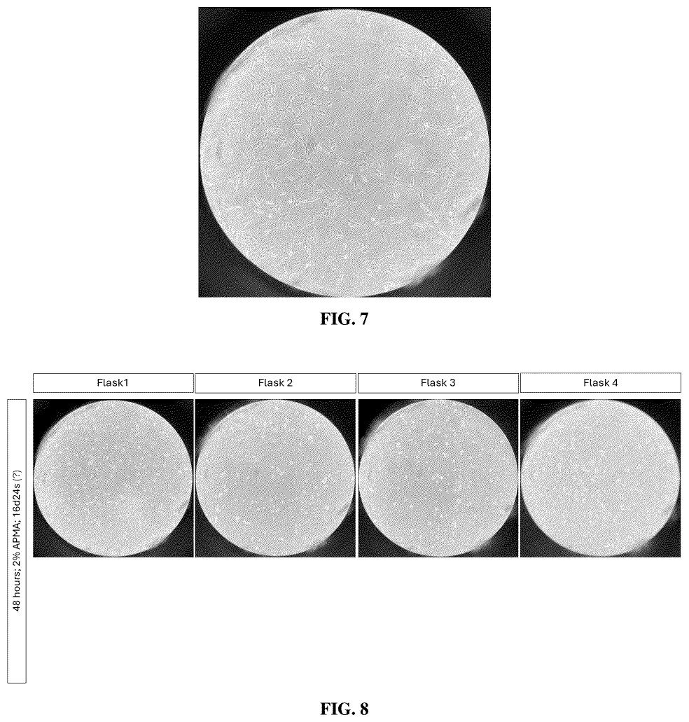

A linear regression model was used to develop a model to predict the adhesion strength above and below the transition temperature for a given percent APMA. This trend shows the ability to tune adhesion strength. This is shown in FIG. 6.

Patterned PNIPAAm surfaces with these properties are currently being developed as an alternative cell culture dish that would allow cells to be detached using the mechanical forces of the PNIPAAm volume phase transition (VPT) temperature instead of chemicals. These polymer surfaces require both strong cell adhesion strength as cells grow in culture above the VPT and for the cells to be able to be released by the mechanical forces created by reducing the temperature to below the VPT. Although the goal is to balance the cell adhesion strength with the ability to release cells below the transition temperature, the ability to maintain cell adhesion strength during a volume phase transition may be useful in other applications, such as selective release (separation) of cell sub populations.

The combination of patterning and lysine-like monomer incorporation creates a tunable increase in adhesion strength both above and below the transition temperature of PNIPAAm. This greatly improves the utility of PNIPAAm which is inherently poorly adhesive. In addition, relatively large increases in adhesion strength above the transition temperature are accompanied by lower levels of adhesion below the transition temperature, indicating weakened adhesion upon triggering the VPT. Further experiments will focus on optimizing the balance between improved cell adhesion on these materials and effective cell release upon triggering the VPT behavior of the surface.

### Example 3: Detachment Experiments

A study was conducted to explore cell detachment. The control was a normal tissue culture polystyrene (TCPS) flask (shown at 48 hours after cell seeding in FIG. 7). The patterned surface had 16 μm polymer features on a TCPS flask. The polymer was a photocrosslinkable thermoresponsive polymer (pNIPAAm) copolymerized with 2% of a cationic monomer. The polymer also included ionic or zwitterionic monomers to see how they modulate cell adhesion prior to the detachment trigger. This copolymer enhances cell adhesion compared to pNIPAAm alone.

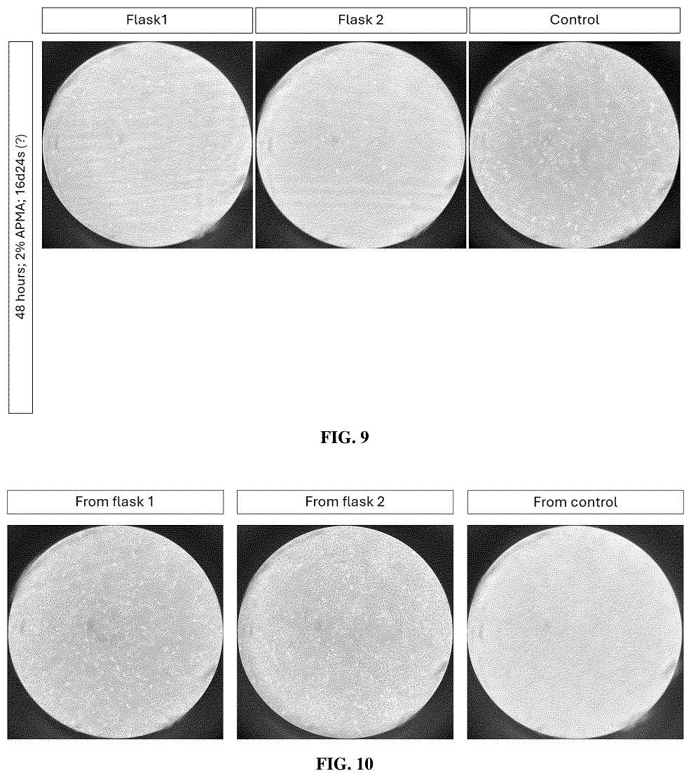

After 48 hours culture, the trigger (reduce temperature by adding cold medium) rapidly expelled the cells from the surface (<1 minute). The flasks before and after detachment are shown in FIG. 8 and FIG. 9, respectively. Control cells did not come off of the control flask even with the same temperature change. Replating detached cells onto normal TCPS after displacement from surfaces showed reattachment and normal morphology 24 hours later (FIG. 10).

When compared with other thermoresponsive polymer systems, detachment happens here very quickly (seconds versus tens of minutes or more) because the mechanism is different. The texture patterns change surface area and shape rapidly as water is taken up so that the cell adhesion is broken, compared to the conventional metabolic response to a change in hydrophobicity.

The following patents, applications and publications as listed below and throughout this document are hereby incorporated by reference in their entirety herein.

What is claimed is:

1. A cell culture substrate, comprising:
a support; and
a copolymer comprising a stimulus-responsive polymer and an ionic polymer;
wherein the copolymer is patterned on the support; and
wherein exposure to a stimulus decreases adhesion strength between the cell culture substrate and a plurality of cells seeded on the cell culture substrate, thereby releasing the plurality of cells from the cell culture substrate.

2. The cell culture substrate of claim 1, wherein the support comprises glass, metal, quartz, tissue culture plastic, or any combination thereof.

3. The cell culture substrate of claim 1, wherein the stimulus-responsive polymer comprises poly(N-isopropylacrylamide) (PNIPAAm), poly(oligo(ethylene glycol) methyl ether methacrylate) (POEGMA), polyethylene oxide (PEO), polyhydroxyethylmethacrylate (PHEMA), poly[2-(dimethylamino)ethyl methacrylate] (PDMAEMA), or any combination thereof.

4. The cell culture substrate of claim 1, wherein the ionic polymer comprises n-(3-aminopropyl) methacrylamide hydrochloride (APMA), acrylic acid, [2-(methacryloyloxy)ethyl]dimethyl-(3-sulfopropyl) ammonium hydroxide, or any combination thereof.

5. The cell culture substrate of claim 1, wherein the copolymer comprises from about 0.1% to about 20% of the ionic polymer.

6. The cell culture substrate of claim 1, wherein the copolymer comprises a photoactivatable crosslinker comprising methacryloyl-4-oxy-benzophenone (MABP), 2,2-dimethoxy-2-phenylacetophenone (DMPA), 2-(4-benzoyl-3-hydroxyphenoxy)ethyl acrylate, or any combination thereof.

7. The cell culture substrate of claim 6, wherein the copolymer comprises from about 1% to about 10% of the photoactivatable crosslinker.

8. The cell culture substrate of claim 1, wherein the stimulus is a change in temperature, pH, an electrical or magnetic field, radiation, ionic concentration, or any combination thereof.

9. The cell culture substrate of claim 8, wherein the stimulus is a change in temperature of from about 1° C. to about 20° C.

10. The cell culture substrate of claim 1, wherein the copolymer increases surface area and/or volume upon exposure to the stimulus, thereby decreasing adhesion strength between the cell culture substrate and the plurality of cells.

11. The cell culture substrate of claim 1, wherein the copolymer pattern comprises a plurality of elements comprising circles, squares, rectangles, hexagons, triangles, or any array or combination thereof.

12. The cell culture substrate of claim 11, wherein each element independently has a surface area of from about 1 μm² to about 500 μm².

13. The cell culture substrate of claim 11, wherein each element is spaced apart from adjacent elements by from about 1 μm to about 50 μm.

14. The cell culture substrate of claim 1, wherein the copolymer covers from about 1% to about 100% of the support.

15. The cell culture substrate of claim 1, wherein the plurality of cells are human.

16. A method of cell culture, the method comprising:
a) providing the cell culture substrate of claim 1;
b) seeding a plurality of cells on the cell culture substrate;
c) exposing the cell culture substrate to a stimulus, thereby releasing the plurality of cells from the cell culture substrate; and
d) collecting the plurality of cells.

17. The method of claim 16, wherein the stimulus is a change in temperature, pH, an electrical or magnetic field, radiation, ionic concentration, or any combination thereof.

18. The method of claim 17, wherein the stimulus is a change in temperature of from about 1° C. to about 20° C.

19. The method of claim 16, wherein from about 50% to about 100% of the plurality of cells are released in from about 1 second to about 300 seconds.

20. The method of claim 16, further comprising:
e) reseeding the plurality of cells on a different substrate.
